(19) United States
(12) Patent Application Publication
(10) Pub. No.: US 2018/0157238 A1
(43) Pub. Date: Jun. 7, 2018

(54) TORQUE SENSOR SAWBLADE ANTI-SKIVING SYSTEM

(71) Applicants: Emily Gogarty, Montreal (CA); Olivier Boisvert, Montreal (CA); Brian M. May, Orange, CT (US); William Hartman, Warsaw, IN (US)

(72) Inventors: Emily Gogarty, Montreal (CA); Olivier Boisvert, Montreal (CA); Brian M. May, Orange, CT (US); William Hartman, Warsaw, IN (US)

(21) Appl. No.: 15/834,476

(22) Filed: Dec. 7, 2017

Related U.S. Application Data
(60) Provisional application No. 62/431,236, filed on Dec. 7, 2016.
(60) Provisional application No. 62/459,194, filed on Feb. 15, 2017.

(51) Int. Cl. G05B 19/406; G05B 19/18; A61B 17/15; A61B 34/00; A61B 90/00; G09B 5/02; G09B 5/04; G09B 9/00

(52) U.S. Cl. CPC G05B 19/406; G05B 19/182; A61B 17/15; A61B 34/76; A61B 90/37; A61B 2017/00022; G09B 5/04; G09B 9/00; G05B 2219/45144; G05B 2219/45123; G05B 2219/36167; G09B 5/02

(57) ABSTRACT

Systems and methods may use a sensor to detect a torque on or from a sawblade. A method may include detecting, using a sensor, a torque on a robotic arm, the torque caused by a sawblade received within a cut guide attached to the robotic arm, generating, in response to receiving a signal from the sensor indicative of the torque on the robotic arm, a visual representation of at least a portion of the torque, and displaying, using a display device, the visual representation of the torque.

### CLAIM OF PRIORITY

This application claims the benefit of U.S. Provisional Patent Application Ser. No. 62/431,236, filed on Dec. 7, 2016, and also claims the benefit of U.S. Provisional Patent Application Ser. No. 62/459,194, filed on Feb. 15, 2017, the benefit of priority of each of which is claimed hereby, and each of which are incorporated by reference herein in its entirety.

### BACKGROUND

A guide is used in surgery to align a cutting, burring, or sawing device with a target object. A cut guide is useful for planning out a cut and allowing for the cut to be precise even in the presence of vibration or movement of the cutting device. However, the cut guide is sometimes not sufficient for ensuring a precise cut due to patient movement, lack of experience, changes in bone density, or obstructed visual access.

Resistance in a bone when being cut by a sawblade, such as due to changes in density or hardness of the bone, may cause skiving, where the sawblade moves in an unexpected or undesirable direction, which results in inaccuracies in the cut. Skiving causes angular movement when the sawblade exits the bone. Current techniques to prevent skiving are imprecise or costly, such as requiring a surgeon to move the sawblade slowly. Also, lasers are sometimes used in industrial applications to prevent or at least identify skiving, but may not be practical in a surgical setting.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 11A-11D illustrate various configurations of a system for detecting torque on a sawblade of a surgical saw in a first view in accordance with some embodiments.

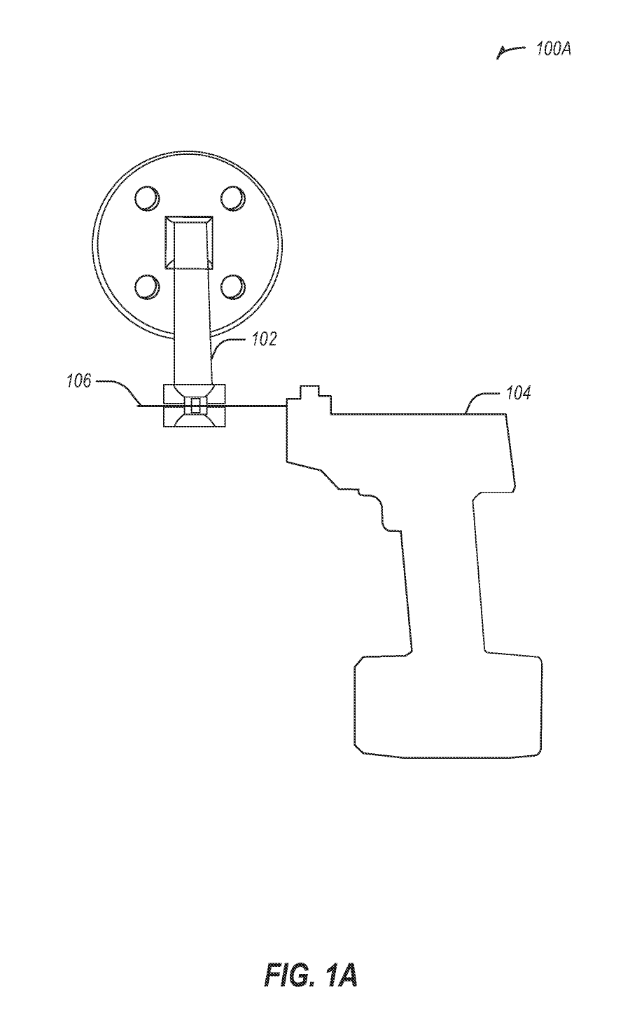

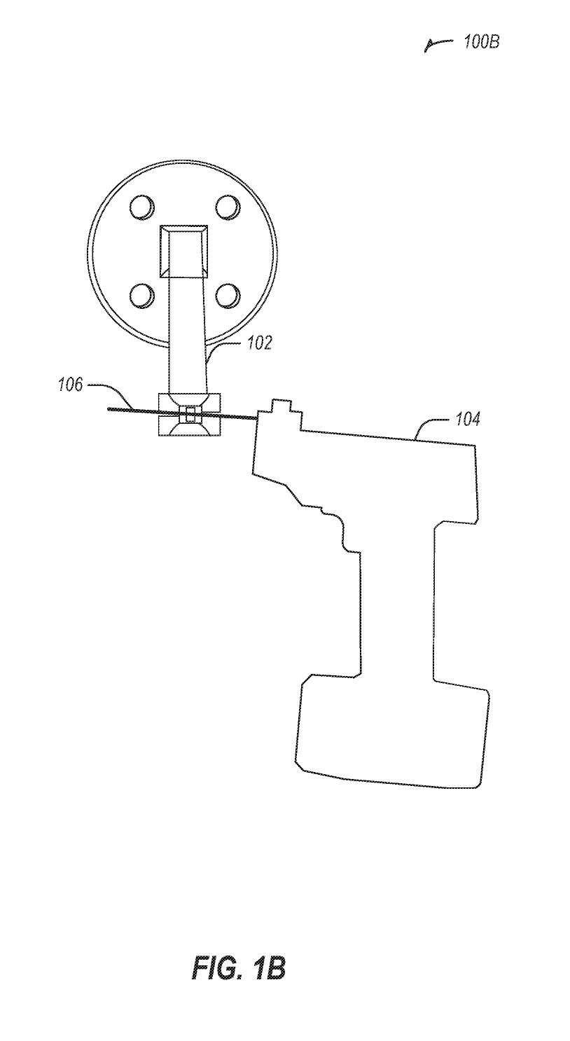

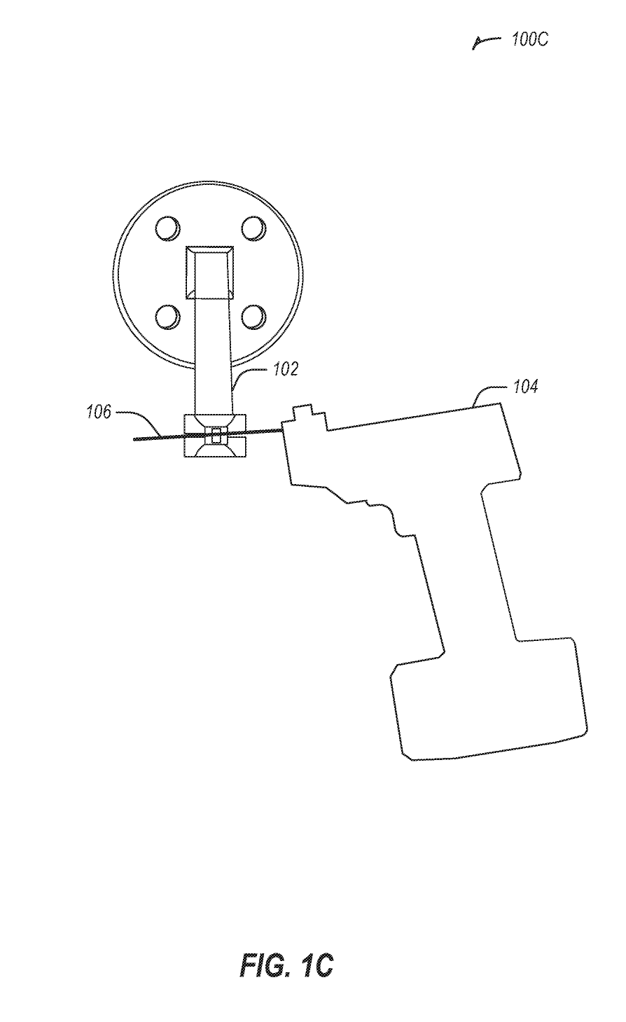

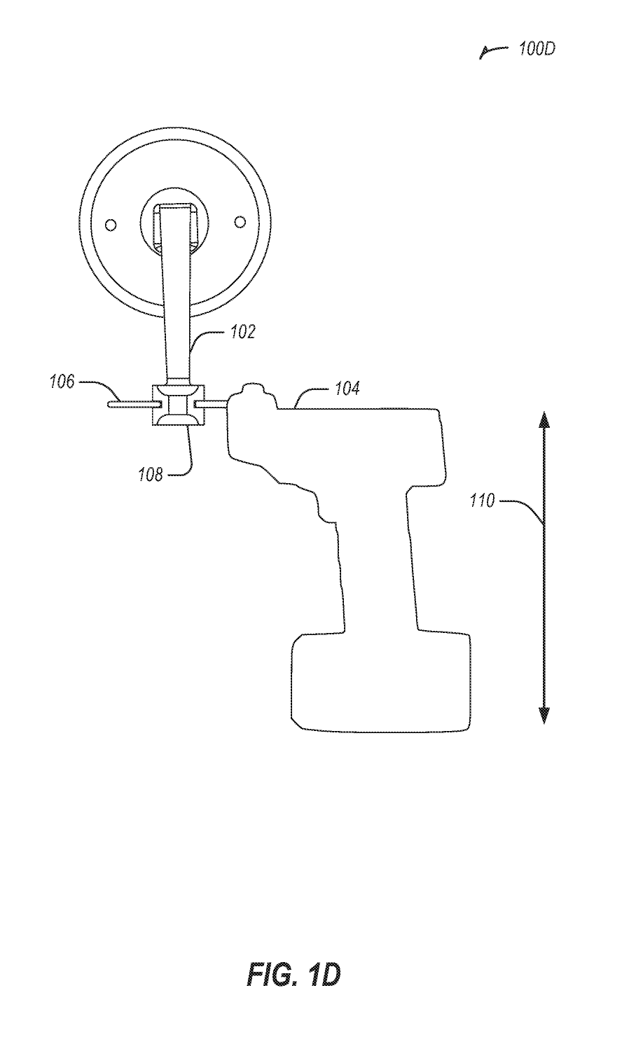

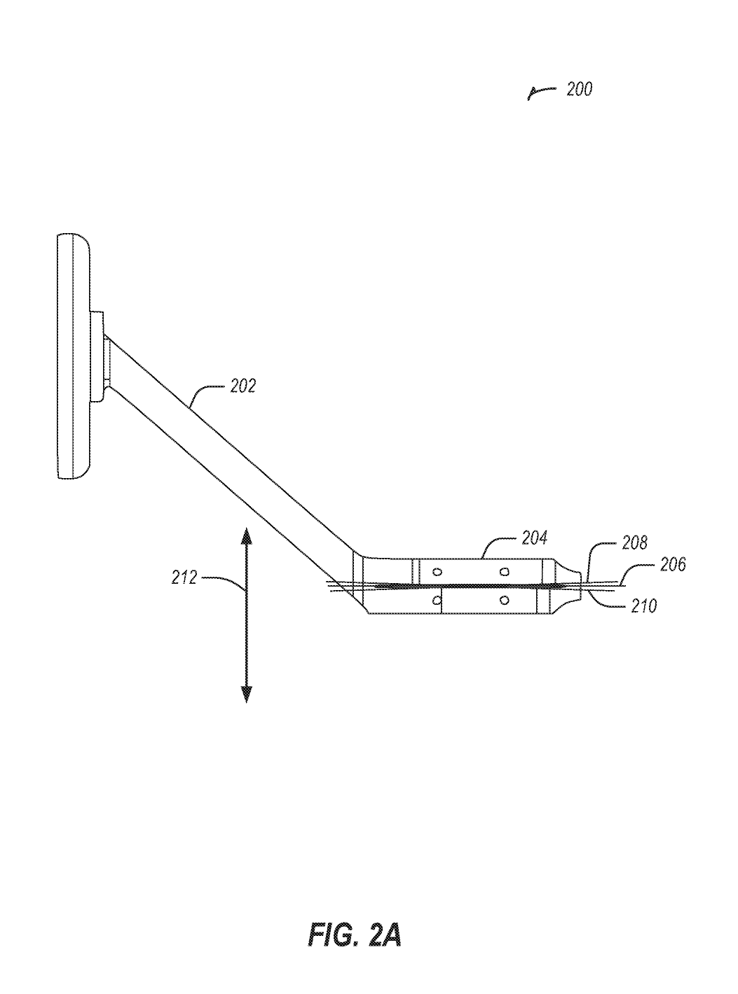

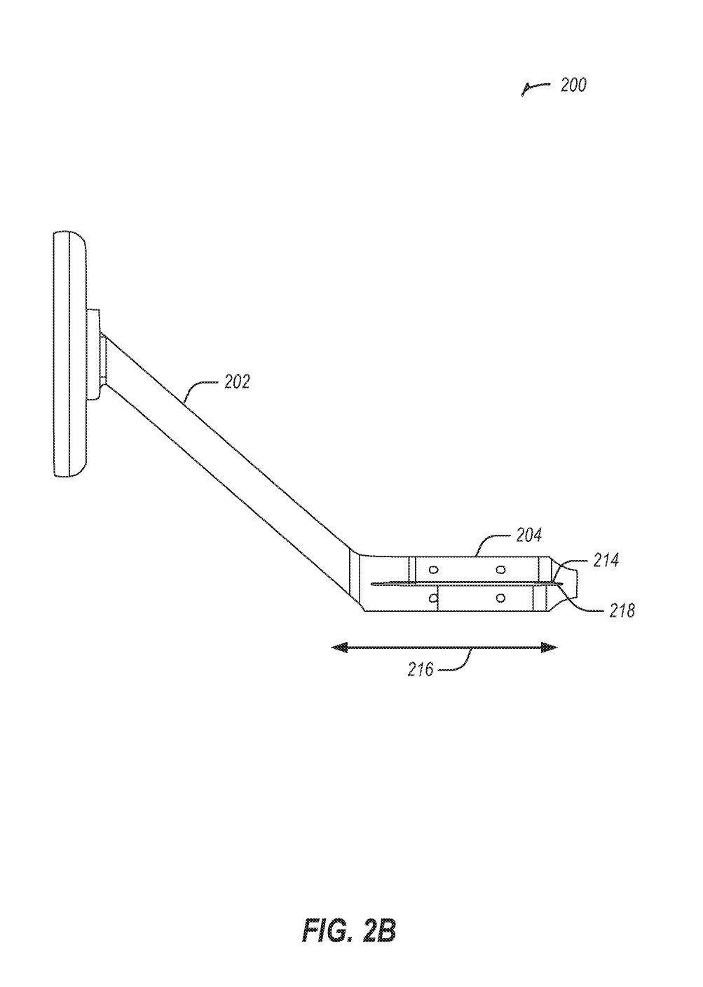

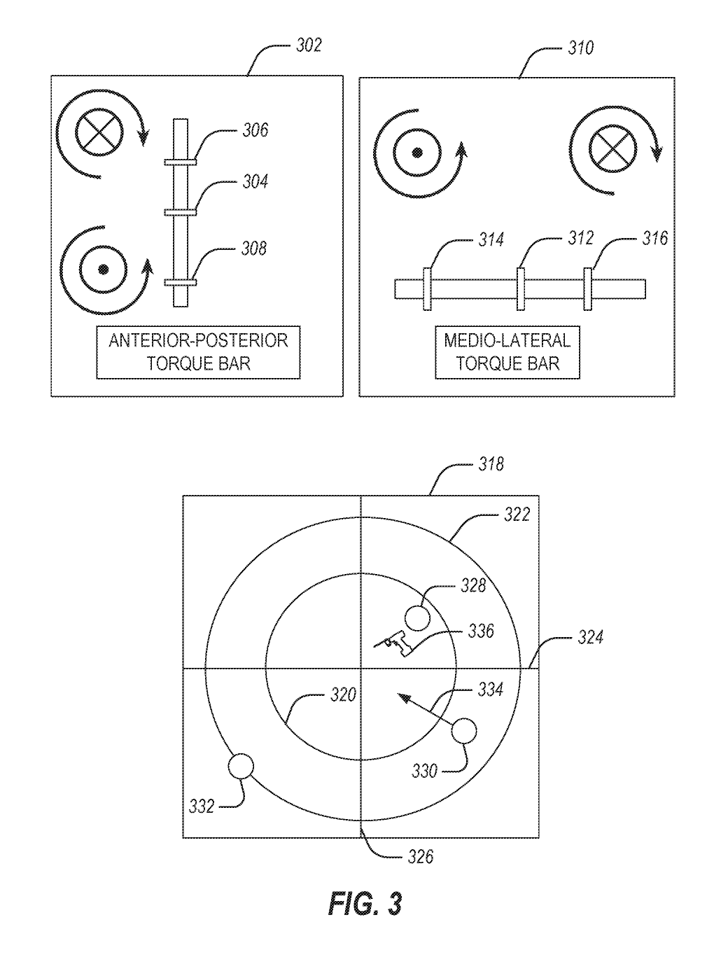

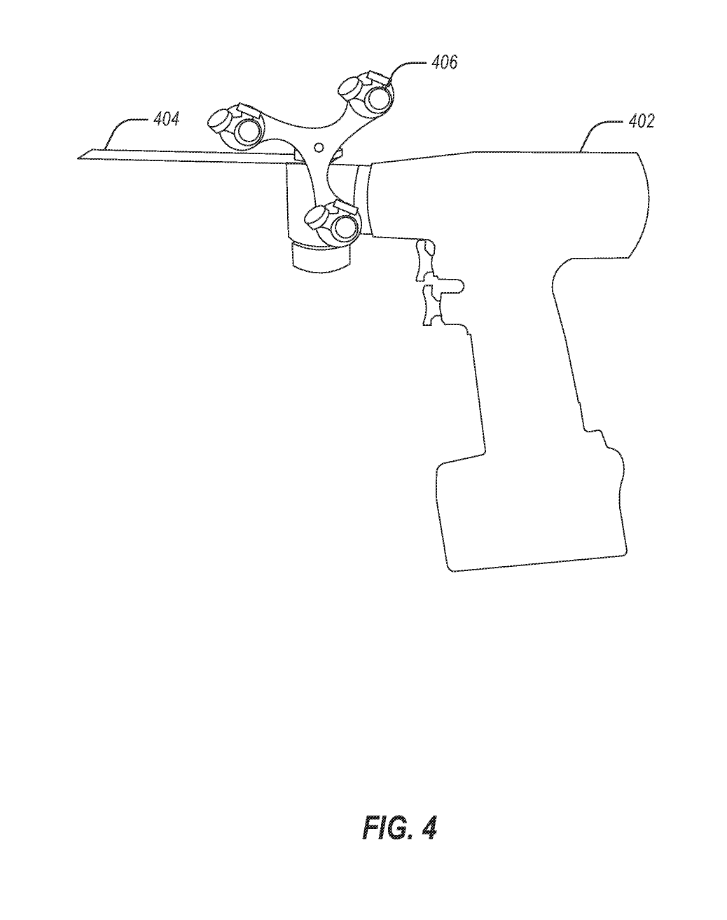

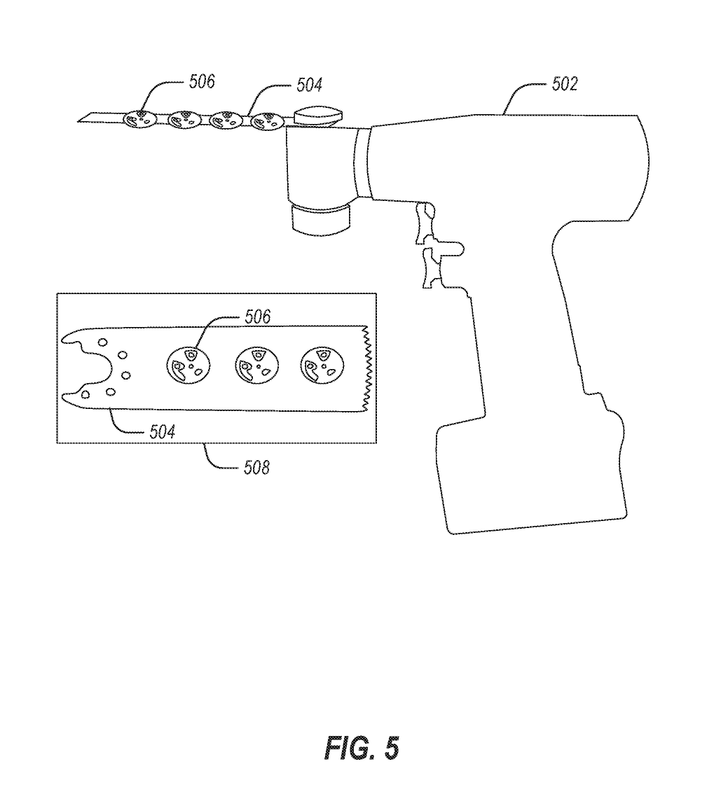

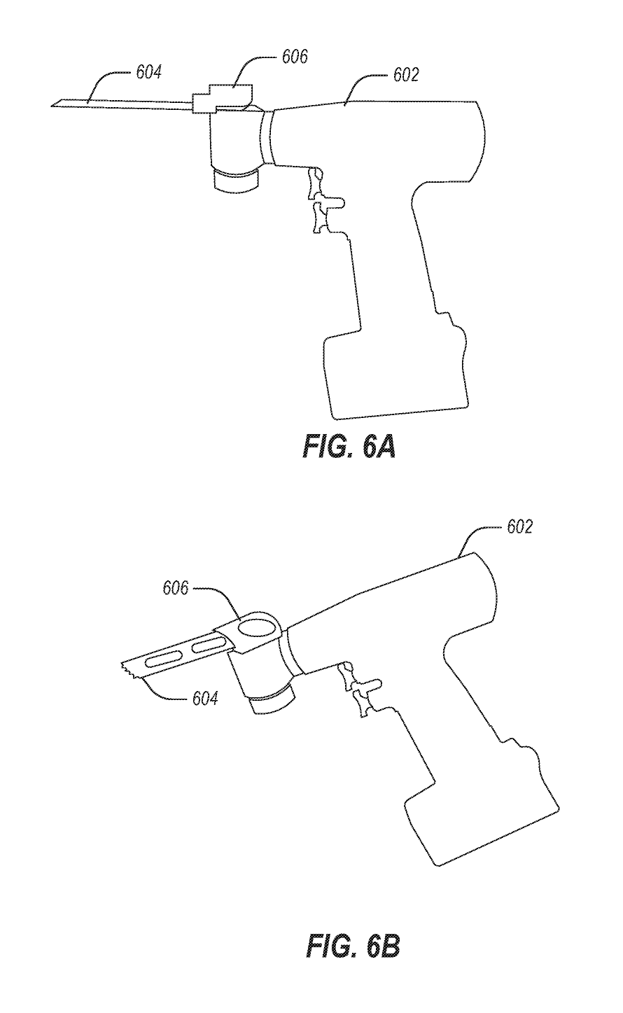

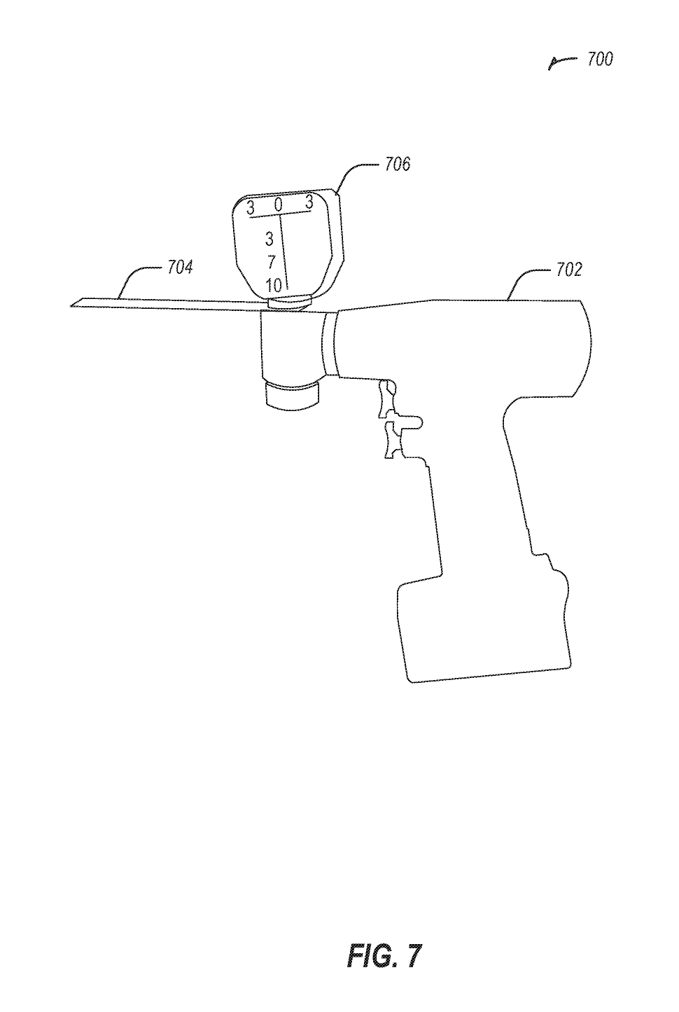

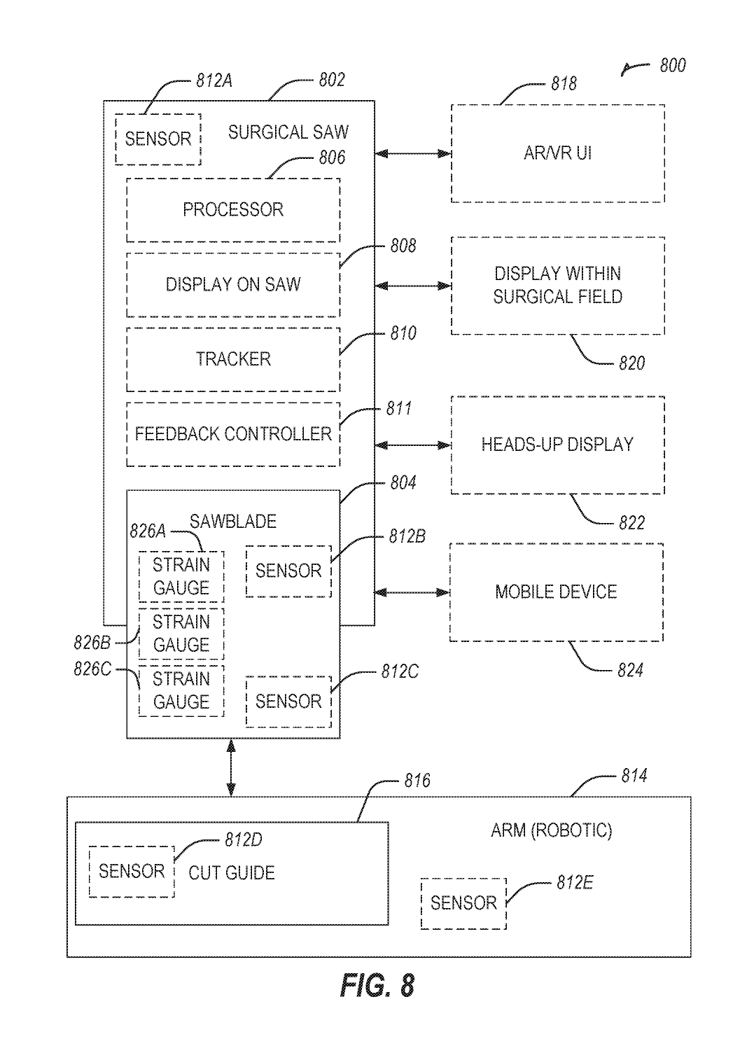

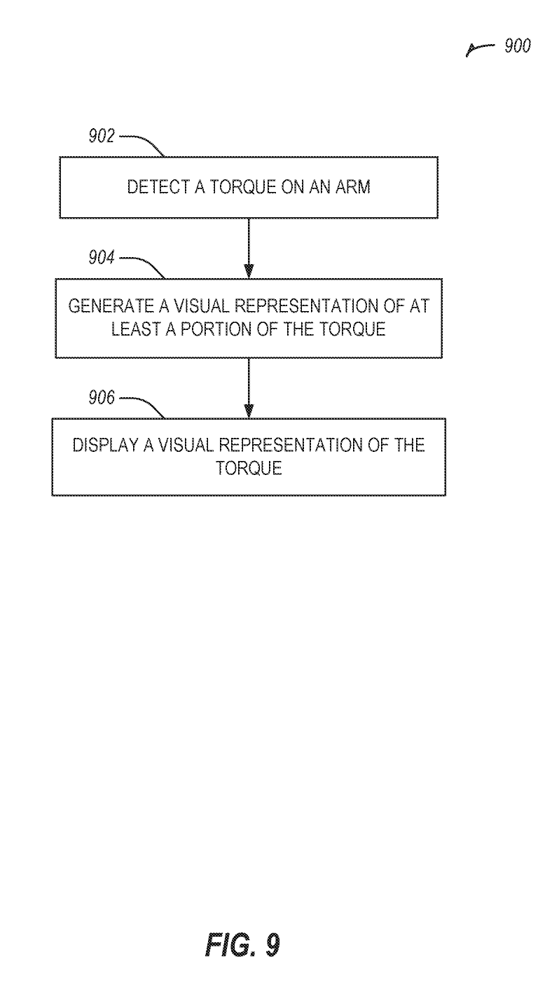

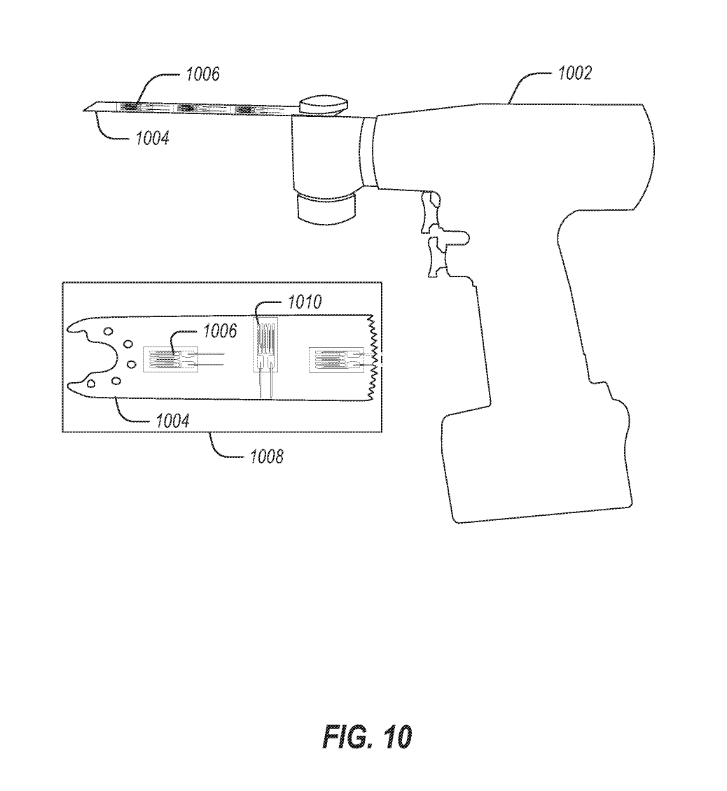

FIGS. 2A-2B illustrate various configurations of a system for detecting torque on a sawblade of a surgical saw in a second view in accordance with some embodiments.

FIG. 3 illustrates various user interfaces for displaying feedback related to torque on a sawblade of a surgical saw in accordance with some embodiments.

FIG. 4 illustrates a surgical saw including a sawblade and a tracker for detecting torque on the sawblade in accordance with some embodiments.

FIG, 5 illustrates a surgical saw including a sawblade and markers on the sawblade for detecting torque on the sawblade in accordance with some embodiments.

FIG. 6A illustrates a side view, and FIG. 6B illustrates an oblique view, of a surgical saw including a sawblade and a force-torque sensor for detecting torque on the sawblade in accordance with some embodiments.

FIG. 7 illustrates a surgical saw including a sawblade and a surgical assistance device for detecting torque on the sawblade in accordance with some embodiments.

FIG. 8 illustrates a system for monitoring a surgical cut with a sawblade in accordance with some embodiments.

FIG. 9 illustrates a flow chart showing a technique for monitoring a surgical cut with a sawblade in accordance with some embodiments.

FIG. 10 illustrates a surgical saw including a sawblade and strain gauges on the sawblade for detecting torque on the sawblade in accordance with some embodiments.

### DETAILED DESCRIPTION

As discussed above, a guide may be used to align a cutting, burring, or sawing device with a target object, such as a target bone. Cut guides are often manually placed by a surgeon on the target object. In other examples, cuts are made using fully or partially autonomous robotic cutting devices. The systems and techniques described herein use a cut guide to guide a sawblade of a surgical saw to make a surgical cut.

Systems and methods for using a surgical saw are described herein. As described above, skiving is a problem that results from a sawblade cutting a bone at an undesirable angle or with an unintended torque. Changes in bone density or hardness may cause skiving, and skiving may also occur with inexperienced surgeons in training. To prevent skiving, the systems and methods described herein provide visual indications of the sawblade, torque on the sawblade or bone, bending or flexing of the sawblade, or skiving occurrences to alert a surgeon to potential skiving issues.

A surgical saw is used to cut bone in various surgical procedures, such as to prepare a bone for receiving an implant or to insert a reinforcement device for setting a broken bone. A cut guide may be used to align a sawblade of the surgical saw with a cutting plane. The sawblade may be placed within the cut guide to perform the cut. The cut guide may include an aperture with tolerance wider than a width of the sawblade, such that the sawblade may move or exert a torque on the cut guide. In another example, a torque applied by the sawblade on the cut guide may cause an anchoring arm of the cut guide to experience a torque. The anchoring arm may include a manual arm, static arm, or a robotic arm. The arm may be locked in place or controlled by a motor. The cut guide may be affixed to a bone (e.g., using screws or glue) or may be relatively free to move in reference to the bone. Generally, a cut guide will be affixed to the bone or held in a constant position relative to the bone by a robotic device.

When a cut guide at the distal end of an arm is used, a sawblade may turn while cutting, causing a torque to be exerted on the arm. Torque exerted by or on a sawblade may be indicative of a potential skiving issue. The torque exerted may be along a medio-lateral plane, along an anterior-posterior plane, a superior-inferior plane, or a combination of one or more planes. The planes are oriented relative to either the patient or the cut guide. In an example, a sensor may be used for a plane to detect torque along that plane. For example, a first sensor may be used to detect a medio-lateral torque and a second sensor may be used to detect an anterior-posterior torque. To detect the torque, various types of sensors may be used, such as an accelerometer, a force sensor, a pressure sensor, or the like. In other examples, a single sensor may be able to detect torque along more than one plane.

When presenting a visual indication or representation (e.g., of a torque, a sawblade, etc.), a display may be used. The display may be mounted on a surgical saw controlling the sawblade, in a surgical field (e.g., on a tablet connected to a gurney or other surgical device such as a lighting device or other display screen), in a virtual reality or augmented reality display, or the like. For example, a light emitting diode (LED) may flash to indicate a torque. In another example, a representation of the sawblade illustrating the torque may be shown on a display screen. Feedback may be displayed, such as a suggested corrective force or action to change the torque. In an example, a virtual cut guide or sawblade may be illustrated in virtual or augmented reality. The virtual cut guide or sawblade may include a color map, such as to show aspects of the cut guide or sawblade that have excessive force or torque applied or where bending or flexing

of the sawblade is occurring. For example, a color map may be overlaid on the actual cut guide or sawblade in augmented reality, or a virtual cut guide or sawblade with colored portions may be shown in virtual reality. An indicator arrow may be displayed in virtual or augmented reality to assist a user in correcting a force, torque, bending, or flexing. For example, an arrow of a corrective force may be displayed to show the user how to move the sawblade to reduce a force, torque, bending, or flexing.

In an example, a torque may be detected in a cut guide attached to a robot arm. For example, when a surgeon applies a force on a sawblade, the force is transferred to the cut guide and to a sensor (e.g., a force or torque sensor), creating a moment of force (torque). A user interface may be used to display the applied forces and guide the surgeon to counter-act the torque and generate a straight cut.

The torques or forces on the sawblades described in FIGS. 1B-1D and 2A-2B may be torques or forces in a single plane or in multiple planes. For example, a rotation of a sawblade may be caused by a rotation or translation of the surgical saw controlling the sawblade. The forces or torques may be caused at one or more points on the sawblade, such as points at which the sawblade is in contact with a cut guide or other portion of an arm, or a point where the sawblade connects to the surgical saw.

FIGS. 1A-1D illustrate various configurations (e.g., 100A-100D) of a system for detecting torque on a sawblade 106 of a surgical saw 104 in a first view in accordance with some embodiments. The position or angle of the surgical saw 104 or the sawblade 106 may be exaggerated in the various configurations 100A-100D for illustrative effect.

FIG. 1A illustrates a first configuration 100A of the first view of the system including the surgical saw 104, the sawblade 106, and an arm 102. The arm 102 may be a manual arm, a static arm, or a robotic arm. The sawblade 106 is illustrated in the first configuration 100A in a position such that the sawblade 106 does not exert a torque (or exerts a minimal torque) on the arm 102. The first configuration 100A may include an ideal position for the sawblade 106 to perform a surgical cut.

FIG. 1B illustrates a second configuration 100B where the sawblade 106 exerts a torque on the arm 102. The first view shows the anterior-posterior plane, and the torque exerted by the sawblade 106 includes an anterior-posterior torque. The torque illustrated in the second configuration 100B, for example, is a torque that would cause the arm 102 to move away from the viewer of FIG. 1B into the page. The torque exerted by the sawblade 106 may not be detectable by a surgeon holding the surgical saw 104 (e.g., due to vibration of the saw, lack of line of sight, etc.). A sensor to detect the torque may be located on the sawblade 106, the cut guide of the robotic arm 102, the robotic arm 102, or the like. In an example, an anterior-posterior torque may be detected independently of other planar torques, and may be displayed together or separately with those other planar torques.

FIG. 1C illustrates a third configuration 100C where the sawblade 106 exerts a torque on the robotic arm 102. The torque exerted by the sawblade 106 in the third configuration 100C may include a torque that would cause the arm 102 to move towards the viewer of FIG. 1C away from the page.

FIG. 1D illustrates a fourth configuration 100D where the surgical saw 104 may move along an axis 110 relative to the arm 102 that may cause a force on the sawblade 106. In this example configuration, the sawblade 106 exerts a torque on the arm 102 from a translational movement of the surgical saw 104 instead of from a rotation of the surgical saw 104 as seen in FIGS. 1B and 1C. The translational movement of the surgical saw 104 may cause a torque, a bending, or a flexing in the sawblade 106 by causing a force on the sawblade 106 at an entrance point or an exit point of the sawblade 106 from a cut guide 108 of the arm 102.

FIGS. 2A-2B illustrate various configurations of a system 200 for detecting torque on a sawblade of a surgical saw in a second view in accordance with some embodiments. FIG. 2A illustrates the system 200 in the second view with an arm 202 and a cut guide 204 and various orientations or angles of a sawblade (e.g., orientations 206-210, exaggerated in FIG. 2 for visualization effect). The second view shows a media-lateral plane of the cut guide 204 and arm 202. The first orientation 206 of the sawblade includes a torque on the cut guide 204, such as a torque that would cause the arm 202 to move toward the viewer of FIG. 2 out of the page. The second orientation 208 of the sawblade illustrates an orientation without a torque on the cut guide 204 or the arm 202 (or with a minimal or negligible torque). The third orientation 210 of the sawblade includes a torque on the cut guide 204, such as a torque that would cause the arm 202 to move away from the viewer of FIG. 2A into the page.

In an example, the sawblade may move in a translational direction along an axis 212 in relation to the cut guide 204. The translational movement of the sawblade may be caused by translational movement of a saw controlling the sawblade. The translational movement may cause a torque, a bending, or a flexing on the sawblade due to interaction with the cut guide 204. The sawblade may be cantilevered to the saw and the cut guide may cause a torque at the connection point of the saw to the sawblade or at the top or bottom of the opening of the cut guide 204.

FIG, 2B illustrates the arm 202, the cut guide 204, and an in-plane orientation 214 of the sawblade. In FIG. 2B, a force may be exerted by the sawblade on the cut guide when the sawblade moves within an opening of the cut guide 204, such as along a cutting plane, and comes into contact with a wall 218 of the opening of the cut guide 204. The contact between the sawblade and the wall 218 while the sawblade maintains the in-plane orientation 214 may cause a force on sawblade from the wall 218. This force may cause friction between the sawblade and the wall 218, reducing accuracy.

To counteract the force and undesirable effects, the arm 202 or the cut guide 204 may be controlled to move along the plane of the in-plane orientation 214. This may prevent the negative effects of the force while leaving the orientation of the sawblade unchanged. A similar force and subsequent movement may occur when the sawblade comes into contact with another wall (e.g., opposite the wall 218) of the cut guide. The movement of the arm 202 or the cut guide 204 may be accompanied by other alerts (e.g., visual, audible, haptic, etc.), or may be performed without alerting the surgeon. The movement of the arm 202 or the cut guide 204 may be automatic, for example, controlled by a motor, control system, or other automated system. The movement may be confined to a range along the plane, for example, a few centimeters of movement in either direction, a predefined range (including, for example, a maximum or minimum range of the arm 202), or the like.

FIG, 3 illustrates various user interfaces (e.g., 302, 310, and 318) for displaying feedback related to torque on a sawblade of a surgical saw in accordance with some embodiments. The user interfaces described with reference to FIG. 3 may be displayed on a saw, on a tablet or mobile device, on a device connected to a surgical gurney, in a virtual or augmented reality display, on heads-display, etc.

A first user interface 302 includes an anterior-posterior torque bar to illustrate torque detected in an anterior-posterior direction. The first user interface 302 includes detected indications 304, 306, and 308, corresponding to a zero torque, a first detected torque, and a second detected torque, respectively. The indication 304 may be used to illustrate when a system has no torque or negligible torque in the anterior-posterior plane. The range of negligible torque may include a range where skiving is unlikely to occur, such as for small torque values. In an example, the anterior-posterior bar may represent a range of possible torques from a maximum outward torque to a maximum inward torque, centered around a zero torque. The extremes of the anterior-posterior bar represent torques that may cause skiving, and the middle of the anterior-posterior bar represents torques that probably will not cause skiving.

The second user interface 310 illustrates a medio-lateral torque bar to represent torques in the medic-lateral plane. The torques in the media-lateral plane may be represented on the medic-lateral bar from an extreme outward torque to an extreme inward torque with a zero torque in the middle. The indication of zero or negligible torque may be represented by indication 312. An outward torque is represented by indication 314 and an inward torque is represented by indication 316.

The first user interface 302 and the second user interface 310 may be displayed separately or may be combined to show both anterior-posterior torque and media-lateral torque together on a single user interface. In another example, a superior-inferior torque bar may be used similarly to the anterior-posterior torque bar or the media-lateral torque bar for displaying indications of torque in the superior-inferior plane. In an example, combinations of any two of these planes may be used to display a two-dimensional representation of torque exerted by a sawblade on an arm (e.g., a robotic arm), such as through a cut guide.

For example, a third user interface 318 is shown in FIG. 3 with a two-dimensional visual representation of torque. In an example, the third user interface 318 illustrates a two-dimensional representation of anterior-posterior and medic-lateral torques. For example, the y-axis 326 may represent the anterior-posterior plane, with an extreme anterior torque at the top and an extreme posterior torque at the bottom of the y-axis 326. The x-axis 324 may represent the medic-lateral plane, with an extreme medial torque at the left and an extreme lateral torque at the right of the x-axis 324.

The third user interface 318 includes, by way of example, a first threshold 322 and a second threshold 320. The first and second thresholds 320 and 322 may be thresholds that, when transgressed, cause an alert or feedback to occur. For example, a warning sound or other audible feedback, a visual indicator such as a flash of a light, or haptic feedback may occur when one or both of the thresholds are transgressed. In another example, the third user interface 318 may. display graduated coloration based on torque level, such as from green around the center of the third user interface 318 to red near the edges of the third user interface 318. The first threshold 320 may correspond with a change in color from more green to more yellow and the second threshold 322 may correspond with a change in color from more yellow to more red. In an example, the first user interface 302 or the second user interface 310 may use color coding to illustrate degree of torque. For example, the bar at the location of the indications 308 or 314 may be red, the bar at the location of the indications 306 or 316 may be yellow, or the bar at the location of the indications 304 or 312 may be green. The bar in the user interfaces 302 or 310 may include graduated coloration, such as from green at the middle of the bar to red near the top or bottom or left or right of the bar.

One or more thresholds e.g.., 320 and 322) may correspond with actions to be taken to control the sawblade. For example, traversing the first threshold 320 may cause the sawblade to slow (e.g., a motor may be controlled to slow the rate of movement of the sawblade). In another example, the first threshold 320 or the second threshold 322 may cause the sawblade to cease cutting. For example, the sawblade may be retracted, a guard may be inserted between the sawblade and a bone being cut, or the sawblade may be stopped, in yet another example, transgressing a threshold may trigger audible, visual, or haptic feedback.

Example indications of detected torque are illustrated in the third user interface 318, although a single torque at a time may be represented. The indications 328, 330, and 332 are shown in various positions in the two-dimensional map, representing different detected torques. For example, a first indication 328 may be central to the first threshold 320, and the first indication 328 may represent a torque that is within normal operating tolerance (although the torque may be causing a slight anterior and lateral force, as indicated by the quadrant location of the first indication 328 within the third user interface 318). The two-dimensional nature of the third user interface 318 allows both anterior-posterior torque and media-lateral torque to be monitored and displayed simultaneously (or a superior-inferior torque may be substituted for one of the other torques). A second indication 330 may represent a torque that has transgressed the first threshold 320 but not the second threshold 322. A risk of skiving may be present with a torque represented by the second indication 330. In an example, the second indication 330 may represent a torque that transgresses the first threshold 320 and causes an alert or feedback to be given to a surgeon, or may cause a sawblade to be slowed down. The second indication 330 may

represent a torque that is not a significant enough skiving risk as to cause the sawblade to be stopped. A third indication 332 may represent a torque that has transgressed both the first and second thresholds 320 and 322. The torque associated with the third indication 332 may cause a significant risk of skiving. The torque detected at this location may cause the sawblade to be stopped or warning feedback to be issued.

In an example, a directional indication may be provided in one of the user interfaces 302, 310, or 318. For example, as shown in the third user interface 318, a directional arrow 334 illustrates a direction to move the surgical saw to decrease undesired torque. The arrow 334 may be dynamic, such as changing direction or length (e.g., showing as a force vector), to indicate changes in direction or amount of force by the undesired torque (e.g., as the undesired torque is decreased by moving in the direction of the arrow 334, the arrow 334 may decrease in length). In another example, a saw representation 336 may be displayed as a directional indication. For example, the drill representation 336 may be animated and move from the first indication 328 towards a center point (i.e., the intersection of the x-axis 324 and the y-axis 326, indicating movement of the surgical saw that should be performed to remove the torque. In another example, the saw representation 336 may have varying length or direction, similar to the arrow 334 described above. In other examples, an indication of a direction to move or a source of undesired torque may be displayed on one of the user interfaces 302, 310, or 318. For example, a text display may indicate the torque is medial or that the surgical saw should be moved in a posterior direction.

In an example, a translation or movement of the cut guide may be caused by a force (e.g., in the distal-proximal plane) applied on the cut guide by the sawblade. A user interface may be used to indicate translation of the cut guide or may show an indication of how to realign the cut guide. In another example, feedback may be issued to alert the surgeon that the cut guide has moved and display a selectable indication, which upon selection may use a robotic arm to automatically move the cut guide back into position.

In an example, a three-dimensional representation of the torque exerted by the sawblade on the arm may be displayed using a user interface. For example, a heads-up or augmented reality display may be used to illustrate the three-dimensional representation of the torque of torque in the anterior-posterior plane, the medio-lateral plane, and the superior-inferior plane simultaneously). In another example, a three-dimensional representation may be displayed or overlaid on the sawblade, saw, or arm to illustrate torque exerted. For example, a torque may be shown with a color component on the cut guide, the sawblade, or the arm.

FIG. 4 illustrates a surgical saw 402 including a sawblade 404 and a tracker 406 for detecting torque on the sawblade in accordance with some embodiments. The tracker 406 may be added to the surgical saw 402 after manufacture, such as just in time for surgery. In another example, the surgical saw 402 may include the tracker 406 integrated at manufacture or added as a permanent or removable component. The tracker 406 may be an attachment compatible with existing surgical saws. In an example, the tracker 406 may be an optical tracker as part of an optical tracking system (e.g., including a remote camera). Other tracking systems may also be used (e.g., ultrasonic).

A tracking system may be used to track location of the tracker 406. The tracker 406 may be connected to the surgical saw 402 at a point of connection between the sawblade 404 and the surgical saw 402. The tracking system may be used to determine when the tracker 406 moves. A second tracker may be placed on a cut guide, or the cut guide may have a location known to the tracking system (e.g., the cut guide may be at a distal end of a robotic arm, the robotic arm may be controlled by the tracking system or in communication with the tracking system). The location of the tracker 406 that is tracked in space may be compared to a previous location or the location of the cut guide. A change in tracker 406 location or movement may be indicative of a torque exerted by the sawblade on the cut guide or an arm the cut guide is attached to. The detected torque may be displayed or the sawblade may be controlled as described herein.

In an example where the cut guide is affixed to the end of a robotic arm the position and orientation of the cut guide may be compared to the known position and orientation of the saw with a tracker. In this example, a user interface may provide positional guidance and induced torque may be derived from a comparison of the saw position and orientation in reference to the cut guide position and orientation. Tracking the saw and cut guide position and orientation will not provide accurate information about torques induced by bone densities or other aspects of the object being cut. Accordingly, position and orientation tracking is best used in conjunction with torque sensors and/or sawblade tracking to provide optimum feedback to the surgeon on quality of the cut.

FIG. 5 illustrates a surgical saw 502 including a sawblade 504 and markers 506) on the sawblade 504 for detecting torque on the sawblade in accordance with some embodiments. FIG. 5 includes a partial, enlarged, top-down view 508 of the sawblade 504 to illustrate marker positions (e.g., marker 506). The markers (e.g., marker 506) may be used to track movement of the sawblade 504. The markers (e.g., marker 506) may be affixed to the sawblade 504 (e.g., as stickers or with glue or magnetically), etched into the sawblade 504, or drawn on the sawblade 504. The markers (e.g., marker 506) may be used similarly to the tracker 406 of FIG. 4, in that the markers (e.g., marker 506) may be tracked using a tracking system (e.g., an optical tracking system). The tracking system may determine movement by the markers (e.g., marker 506) and detect a torque based on the movement.

In an example, some torque sensors may react to find bending or torque at the base of a sawblade. By placing the markers (e.g., marker 506) directly on the sawblade 504, the sawblade itself may be monitored for torque, rather than just at the base of the sawblade 504. Using markers on the sawblade 504 may allow for tracking bending throughout the sawblade 504 rather than just the base, which may be more precise or identify torques that are not apparent at the base.

FIG. 6A illustrates a side view, and FIG. 6B illustrates an oblique view, of a surgical saw 602 including a sawblade 604 and a force-torque sensor 606 for detecting torque on the sawblade 604 in accordance with some embodiments. In an example, the force-torque sensor 606 may be detachable from the surgical saw 602. The force-torque sensor 606 may be compatible with existing surgical saws and added after manufacture. In another example, the force-torque sensor 606 may be integrated with the surgical saw 602 at manufacture. The force-torque sensor 606 may be used to detect torque on the sawblade 604 (e.g., a reciprocal force of the force exerted by the sawblade 604 on a cut guide). The torque detected by the force-torque sensor 606 may be used to provide feedback, stop the sawblade 604, etc., as described herein.

In an example, the sensor 606 may include a multi-axis (e.g., a 6-axis) force torque sensor. The sensor 606 may be integrated at the end of a robotic arm, or between a robotic arm and an end effector (e.g., cut guide). In another example, the sensor 606 may be positioned between the sawblade 604 and a handle of the surgical saw 602. The force torque sensor may include one or more strain gauges to measure force or torque in a direction. A plurality of strain gauges may be included in the sensor 606 to measure forces or torques in a plurality of respective directions. The force or torque measured by the sensor 606 may be related to force or torque applied to the sawblade 604 or the surgical saw 602, and may be determined based on known geometry of the system.

In an example, one or more strain gauges may be included on or in the sawblade 604 to detect bending or flexing of the sawblade. The one or more strain gauges may be linked in a mesh for coverage of aspects of the sawblade 604. A warning indicator may be displayed via one of the user interface elements described herein to alert a user to excessive bending or flexing of the sawblade 604 or an indication of a degree of bending or flexing in the sawblade 604 may be displayed, audibly indicated, or the like. When the bending or flexing exceeds one or more configurable thresholds, actions may be taken to control the sawblade 604 as described herein (e.g., automatically moving a cut guide, end effector, or robotic arm, stopping the sawblade 604, etc.). The detected bending or flexing of the sawblade may be used by a tracking system to correct an optically tracked position of the sawblade 604, or the tracking systems described in FIG. 4, :5, or 7. The detected bending or flexing may be displayed, such as using an augmented reality display, which may indicate or suggest a direction or an action to correct the bending or flexing of the sawblade 606, such as by using a virtual model of the sawblade 606.

FIG. 7 illustrates a surgical saw 702 including a sawblade 704 and a surgical assistance device 706 for detecting torque on the sawblade 704 in accordance with some embodiments. The surgical assistance device 706 may include the iAssist from Zimmer of Warsaw, Ind. for total knee arthroplasty. The iAssist includes a display that may be attached to the surgical saw 702. In an example the surgical assistance device 706 may be used in combination with a sensor or technique described herein to detect torque and display a visual representation of the torque on the display of the surgical assistance device 706. The iAssist is a device that includes an electronic guidance system that may use inertial navigation to determine location, movement, force, etc. The iAssist may be used for a knee surgical procedure, such as to help a surgeon align or validate bony resections in real-time within a surgical field. In :FIG. 7, the iAssist may be used to track location of the surgical saw 702 or the sawblade 704, as well as measure or detect torque on the sawblade 704, such as by using inertial sensors within the iAssist.

The surgical assistance device 706 may be used to determine a relative orientation of the sawblade 704 to a cut guide. An accelerometer or gyroscope based technology in the surgical assistance device 706 may be used to determine whether there is a difference in orientation of the cut guide or a cut plane to a plane of the sawblade 704. In response to determining that there is a difference in orientation, the surgical assistance device 706 may suggest a correction to a surgeon operating the surgical saw 702, such as by presenting visual, audible, or haptic feedback. For example, the angle difference determined may be displayed in a user interface, or on the surgical assistance device 706 itself.

FIG. 8 illustrates a system 800 for monitoring a surgical cut with a sawblade in accordance with some embodiments. The system 800 includes a surgical saw 802, which includes a sawblade 804. The sawblade 804 may fit into a cut guide 816, which may be affixed to a distal end of an arm 814, such as a robotic arm. The surgical saw 802 may include a processor 806 to determine whether a torque transgresses a threshold. In an example, a processor may be remote to the surgical saw 802 and in communication with a component of the surgical saw 802, such as a sensor 812A. The processor 806 may be used to control operation of the surgical saw 802 in response to signals from the sensor (e.g., 812A-E indicative of torque on the sawblade 804. The processor 806 may determine whether the detected torque transgressed a threshold, and to control operation of the surgical saw, the processor 806 may control operation of the surgical saw 804 in response to determining that the detected torque transgressed the threshold. For example, the processor 806 may cause the sawblade 804 to cease operation in response to determining that the detected torque transgressed the threshold. Ceasing operation of the sawblade 804 may be accomplished by inserting a guard, causing a motor to stop the sawblade 804 or retracting the sawblade 804 into a shaft of the surgical saw 802.

Sensors (e.g., 812A) may be located on the surgical saw 802, on a sawblade (e.g., 812B, on a portion of the saw/blade 804 at a location where the sawblade 804 connects to the surgical saw 802, or 812C, on a portion of the sawblade 804 extending from the surgical saw 802), on the cut guide 816 (e.g., 812D), or on the arm 814 (e.g., 812E). The various sensors 812A-E may be used to detect torque, for example torque exerted by the sawblade 804 on the arm 814 (e.g., via the cut guide 816) or a torque resulting from the equal and opposite force of the force that caused that torque. Multiple sensors may be used, such as to detect torque in different planes or to detect orthogonal torques. For example, the sensor 812E may include two or more sensors for detecting torques, such as an anterior-posterior torque and a medio-lateral torque. The sensors 812A-E may include an accelerometer, a force sensor, or a pressure sensor.

In another example, one or more strain gauges (e.g., 826A-C) may be located on the sawblade 804. The strain gauges 826A-C may be used to detect torque, for example torque exerted directly on a portion of the sawblade 804, which may not be detectable at the base of the sawblade 804 or at the surgical saw 802. Multiple strain gauges may be used, such as to detect torque in different planes, along different axes, or to detect orthogonal torques. For example, the strain gauge 826A may detect an anterior-posterior torque and strain gauge 826B may detect a medio-lateral torque.

Sensor data from one or more of the sensors 812A-E may be used to display an indication or visual representation of the torque. A representation of the sawblade 804 or surgical saw 802 may also be displayed. An alert may be displayed in response to the processor 806 determining that the detected torque has transgressed a threshold. The indications, visual representations, alerts, or sawblade 804 representation may be shown on a display on the surgical saw 808, an augmented reality or virtual reality user interface 818, a display within the surgical field 820 (e.g., a tablet), or a heads-up display 822. The visual indication displayed on one or more of these optional displays may include an anterior-posterior torque bar corresponding to an anterior-posterior detected torque, a medio-lateral torque bar corresponding to a medio-lateral detected torque, or a an anterior-posterior torque axis corresponding to the anterior-posterior detected torque and a medic-lateral torque axis corresponding to the medic-lateral detected torque in a two-dimensional torque map. A visual indication may include a representation of the detected torque with a visual depiction of an orientation of the sawblade 804 relative to the cut guide 816.

The surgical saw 802 may include a feedback controller 811 to control feedback presented to a surgeon or a student raining to be a surgeon. The feedback controller 811 may control visual feedback, audio feedback, haptic feedback, or the like. The surgical saw 802 may be used to make a training cut, performed using the sawblade 804, by a student. In the student setup, the surgical saw 802 may use the feedback controller 811 to provide feedback indicative of a corrective action to be taken to change the detected torque, such as qualitative feedback (e.g., “tilt the surgical saw down”), a visual indication (e.g., flashing a light on a display, such as the display on the surgical saw 808), an auditory indication (e.g., a beep or vocalized feedback), or haptic feedback (e.g., causing the surgical saw 802 to vibrate when a torque transgresses the threshold which may not be a useful feature when not in the student setup). In an example, the surgical saw 802 may provide feedback using the feedback controller 811 related to a bending or flexing of the sawblade 804. For example, a visual indication of the bending or flexing may be displayed (e.g., using the heads-up display 822, the display within the surgical field 802, the AR/VI UI 818, or the display on the surgical saw 808) or a corrective action or suggestion may be displayed. In another example, audible feedback, haptic feedback, or the like may be output in response to detecting a bending or flexing of the sawblade 804.

In an example, the AR/VR UI 818 may be used to display feedback, such as related to force, torque, bending, or flexing on the sawblade 804. For example, a virtual cut guide or sawblade may be illustrated in virtual or augmented reality using the AR/VR UI 818. The virtual cut guide or sawblade may include a color map, such as to show aspects of the cut guide or sawblade that have excessive force or torque applied or where bending or flexing of the sawblade is occurring. For example, a color map may be overlaid on the actual cut guide or sawblade in augmented reality, or a virtual cut guide or sawblade with colored portions may be shown in virtual reality using the AR/VR UI 818. An indicator arrow may be displayed in virtual or augmented reality on the AR/VR UT 818 to assist a user in correcting a force, torque, bending, or flexing. For example, an arrow of a corrective force may be displayed to show the user how to move the sawblade to reduce a force, torque, bending, or flexing.

A component of the surgical saw 802 (such as the processor 806 or the sensor 812A) may be in communication with a device 824 (e.g., a computer or a mobile device), which may include a processor and display screen. The device 824 may be used to receive signals from a sensor (e.g., 812A-E) indicative of the detected torque. The device 824 may generate a visual indication representative of the detected torque for display on the device. In an example, the device 824 is affixed to the surgical saw 802. In an example, the device 824 may determine, based on the received signals, whether the detected torque transgressed a threshold. The device 824 may, in response to determining that the detected torque transgressed the threshold, present a visual indication that the detected torque transgressed the threshold (e.g., on the display of the device 824). In another example, the device 824 is remote from the surgical saw 802, and used in a surgical field.

FIG. 9 illustrates a flow chart showing a technique 900 for monitoring a surgical cut with a sawblade in accordance with some embodiments. The technique 900 includes an operation 502 to detect a torque on an arm (e.g., a robotic arm). The torque may be detected using a sensor, and the detected torque may be caused by a sawblade and received within a cut guide attached to the arm (which may include a robotic arm). The technique 900 includes an operation 904 to generate a visual representation of at least a portion of the torque. Operation 904 may be generated in response to receiving a signal from the sensor indicative of the torque on the arm.

The technique 900 includes an operation 906 to display the visual representation of the torque, such as by using a display device. Displaying the visual representation may include displaying an anterior-posterior torque bar corresponding to an anterior-posterior detected torque or a media-lateral torque bar corresponding to a media-lateral detected torque or displaying a representation of the detected torque within a two-dimensional torque map. Displaying the two-dimensional torque map may include displaying torque components representing a medio-lateral detected torque and an anterior-posterior detected torque. The two-dimensional torque map may be displayed using a graduated coloration, such as based on torque level. The graduated coloration may change from green to red (e.g., green in the center of the map and red at the edges). In an example, the visual representation may be displayed on at least one of a display in a surgical field, a virtual reality display, an augmented reality display, or a heads-up display. In another example, the display device may be located on a surgical saw controlling the sawblade.

The technique 900 may include an operation to determine whether the detected torque transgressed a threshold, and controlling operation of the surgical saw in response to determining that the detected torque transgressed the threshold. Controlling operation of the surgical saw may include causing the sawblade to cease operation in response to determining that the detected torque transgressed the threshold. In an example, in response to determining that the torque transgressed the threshold, the technique 900 may include stopping the sawblade, retracting the sawblade, or blocking the sawblade, such as with a guard.

FIG. 10 illustrates a surgical saw 1002 including a sawblade 1004 and strain gauges (e.g., 1006) on the sawblade 1004 for detecting torque on the sawblade 1004 in accordance with some embodiments. FIG. 10 includes a partial, enlarged, top-down view 1008 of the sawblade 1004 to illustrate strain gauge positions (e.g., strain gauge 1006). The strain gauge (e.g., strain gauge 1006) may be used to track movement of the sawblade 1004. The strain gauge (e.g., strain gauge 1006) may be affixed or coupled to the sawblade 1004 (e.g., as stickers or with glue or magnetically). The strain gauge (e.g., strain gauge 1006) may output strain (e.g., as a voltage, value, etc.) to a processor, memory, via a transceiver or transmitter to a remote device (e.g., the surgical saw 1002 or a computer), or the like. The strain gauges may be oriented along different axes to measure different strains, such as along a long axis of the sawblade 1004 (e.g., strain gauge 1006), along a short axis of the sawblade 1004 (e.g., strain gauge 1010), along a 45 degree axis to one of the short or long axis, or any other angle desired by the surgeon when measuring potential torque on the sawblade 1004.

In an example, some torque sensors may react to find bending or torque at the base of a sawblade. By placing the strain gauge (e.g., strain gauge 1006) directly on the sawblade 1004, the sawblade 1004 itself may be monitored for torque, rather than just at the base of the sawblade 1004. Using strain gauges on the sawblade 1004 may allow for tracking strain or bending throughout the sawblade 1004 rather than just the base, which may be more precise or identify torques that are not apparent at the base.

Sawblades, such as sawblade 1006, may be used in conjunction with or instead of the various method and apparatus for detecting skiving discussed above. For example, technique 900 may leverage information derived from sawblade 1006 to display sawblade torque information and/or alert a surgeon of potential skiving. In an example, the sawblade 1006 may be used with system 800, for example as sawblade 804, including strain gauges 826A-C.

In an example the term “machine readable medium” may include a single medium or multiple media (e.g., a centralized or distributed database, or associated caches and servers) configured to store one or more instructions. The term “machine readable medium” may include any medium that is capable of storing, encoding, or carrying instructions for execution by a machine and that cause the machine to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks,

### VARIOUS NOTES & EXAMPLES

Each of these non-limiting examples may stand on its own, or may he combined in various permutations or combinations with one or more of the other examples.

Example 1 is a surgical cutting system comprising: a surgical saw including a sawblade to perform a surgical cut; a cut guide used to guide the sawblade while performing the surgical cut; a sensor to detect torque on the sawblade induced by interaction with the cut guide during the surgical cut; and a device including a processor and display screen configured to: receive signals from the sensor indicative of the detected torque; and generate a visual indication representative of the detected torque for display on the device.

In Example 2, the subject matter of Example 1 includes, wherein the device is affixed to the surgical saw.

In Example 3, the subject matter of Examples 1-2 includes, wherein the device is further configured to determine, based on the received signals, whether the detected torque transgressed a threshold.

In Example 4, the subject matter of Example 3 includes, wherein the device is further configured to, in response to determining that the detected torque transgressed the threshold, present a visual indication that the detected torque transgressed the threshold.

In Example 5, the subject matter of Examples 1-4 includes, wherein the visual indication includes an anterior-posterior torque bar corresponding to an anterior-posterior detected torque or a medio-lateral torque bar corresponding to a medio-lateral detected torque.

In Example 6, the subject matter of Examples 1-5 includes, a second sensor to detect torque in a direction orthogonal to the sensor.

In Example 7, the subject matter of Example 6 includes, wherein the sensor detects an anterior-posterior torque and the second sensor detects a medio-lateral torque.

In Example 8, the subject matter of Examples 6-7 includes, wherein the visual indication includes an anterior-posterior torque axis corresponding to the anterior-posterior detected torque and a medio-lateral torque axis corresponding to the medio-lateral detected torque in a two-dimensional torque map.

In Example 9, the subject matter of Examples 1-8 includes, wherein the device is remote from the surgical saw, and used in a surgical field.

In Example 10, the subject matter of Examples 1-9 includes, wherein the sensor is positioned within the cut guide.

In Example 11, the subject matter of Examples 1-10 includes, wherein the display is a virtual reality display, augmented reality display, or a heads-up display.

In Example 12, the subject matter of Examples 1-11 includes, wherein the display is located on the surgical saw.

In Example 13, the subject matter of Examples 1-12 includes, wherein the sensor is one of an accelerometer, a force sensor, or a pressure sensor, or a strain gauge.

In Example 14, the subject matter of Examples 1-13 includes, wherein the visual indication representative of the detected torque includes a visual depiction of an orientation of the sawblade relative to the cut guide.

Example 15 is a surgical saw comprising: a sawblade to perform a surgical cut using a cut guide; a sensor positioned on the sawblade, the sensor to detect torque on the sawblade by the cut guide during the surgical cut; a processor to control operation of the surgical saw in response to signals from the sensor indicative of torque on the sawblade.

In Example 16, the subject matter of Example 15 includes, wherein the processor is further to determine whether the detected torque transgressed a threshold, and wherein to control operation of the surgical saw, the processor is to control operation of the surgical saw in response to determining that the detected torque transgressed the threshold.

In Example 17, the subject matter of Example 16 includes, wherein to control operation of the surgical saw includes to cause the sawblade to cease operation in response to determining that the detected torque transgressed the threshold.

In Example 18, the subject matter of Example 17 includes, wherein to cause the sawblade to cease operation, the processor is to cause a guard to block the sawblade.

In Example 19, the subject matter of Examples 17-18 includes, wherein to cause the sawblade to cease operation, the processor is to cause a motor to stop the sawblade.

In Example 20, the subject matter of Examples 17-19 includes, wherein to cause the sawblade to cease operation, the processor is to cause the sawblade to be retracted into a shaft of the surgical saw.

In Example 21, the subject matter of Examples 16-20 includes, wherein the surgical saw further comprises a display to, in response to the processor determining that the detected

torque exceeded the threshold, present a visual indication that the detected torque transgressed the threshold.

In Example 22, the subject matter of Examples 16-21 includes, wherein the processor is further to generate a visual indication representative of the detected torque for display on a display screen.

In Example 23, the subject matter of Example 22 includes, wherein the visual indication includes an anterior-posterior torque bar corresponding to an anterior-posterior detected torque or a media-lateral torque bar corresponding to a media-lateral detected torque.

In Example 24, the subject matter of Examples 22-23 includes, wherein the visual indication includes a representation of the detected torque within a two dimensional torque map.

In Example 25, the subject matter of Examples 22-24 includes, wherein display screen is located on one of a mobile device, the sawblade, or the surgical saw.

In Example 26, the subject matter of Examples 15-25 includes, wherein the sensor is one of an accelerometer, a force sensor, or a pressure sensor, or a strain gauge.

In Example 27, the subject matter of Examples 15-26 includes, wherein the surgical cut is a training cut performed using the sawblade by a student and wherein the surgical saw further comprises a feedback component to provide feedback indicative of the detected torque.

In Example 28, the subject matter of Example 27 includes, wherein the feedback includes at least one of a visual indication, an auditory indication, or haptic feedback.

In Example 29, the subject matter of Examples 15-28 includes, wherein the sawblade includes a plurality of strain gauges, located on the sawblade, to determine a bending or flexing in the sawblade and wherein the processor is further to control operation of the surgical saw in response to the determined bending or flexing.

Example 30 is a method comprising: detecting, using a sensor, a torque on a robotic arm, the torque caused by a sawblade received within a cut guide attached to the robotic arm; generating, in response to receiving a signal from the sensor indicative of the torque on the robotic arm, a visual representation of at least a portion of the torque; and displaying, using a display device, the visual representation of the torque.

In Example 31, the subject matter of Example 30 includes, wherein displaying the visual representation of the torque includes displaying an anterior-posterior torque bar corresponding to an anterior-posterior detected torque or a medio-lateral torque bar corresponding to a medio-lateral detected torque.

In Example 32, the subject matter of Examples 30-31 includes, wherein displaying the visual representation of the torque includes displaying a representation of the detected torque within a two-dimensional torque map.

In Example 33, the subject matter of Example 32 includes, wherein displaying the two-dimensional torque map includes displaying torque components representing a media-lateral detected torque and an anterior-posterior detected torque.

In Example 34, the subject matter of Examples 32-33 includes, wherein displaying the two-dimensional torque map includes displaying graduated coloration based on torque level.

In Example 35, the subject matter of Example 34 includes, wherein the graduated coloration varies from green to red.

In Example 36, the subject matter of Examples 30-35 includes, wherein displaying the visual representation of the torque includes displaying the visual representation on at least one of a display in a surgical field, a virtual reality display, an augmented reality display, or a heads-up display.

In Example 37, the subject matter of Examples 30-36 includes, wherein the display device is located on a surgical saw controlling the sawblade.

In Example 38, the subject matter of Examples 30-37 includes, determining whether the detected torque transgressed a threshold, and controlling operation of the surgical saw in response to determining that the detected torque transgressed the threshold,

In Example 39, the subject matter of Example 38 includes, wherein controlling operation of the surgical saw includes causing the sawblade to cease operation in response to determining that the detected torque transgressed the threshold.

In Example 40, the subject matter of Examples 38-39 includes, stopping the sawblade in response to determining that the torque transgressed the threshold.

In Example 41, the subject matter of Examples 38-40 includes, retracting the sawblade in response to determining that the torque transgressed the threshold.

In Example 42, the subject matter of Examples 38-41 includes, blocking the sawblade with a guard component in response to determining that the torque transgressed the threshold.

Example 43 is at least one machine-readable medium including instructions for operation of a surgical saw, which executed by a processor, cause the processor to perform operations to: detect, using a sensor, a torque on a robotic arm, the torque caused by a sawblade received within a cut guide attached to the robotic arm; generate, in response to receiving a signal from the sensor indicative of the torque on the robotic arm, a visual representation of at least a portion of the torque; and display, using a display device, the visual representation of the torque.

In Example 44, the subject matter of Example 43 includes, wherein displaying the visual representation of the torque includes displaying an anterior-posterior torque bar corresponding to an anterior-posterior detected torque or a medio-lateral torque bar corresponding to a media-lateral detected torque.

In Example 45, the subject matter of Examples 43-44 includes, wherein displaying the visual representation of the torque includes displaying a representation of the detected torque within a two-dimensional torque map.

In Example 46, the subject matter of Example 45 includes, wherein displaying the two-dimensional torque map includes displaying torque components representing a medio-lateral detected torque and an anterior-posterior detected torque.

In Example 47, the subject matter of Examples 45-46 includes, wherein displaying the two-dimensional torque map includes displaying graduated coloration based on torque level.

In Example 48, the subject matter of Example 47 includes, wherein the graduated coloration varies from green to red.

In Example 49, the subject matter of Examples 43-48 includes, wherein displaying the visual representation of the torque includes displaying the visual representation on at least one of a display in a surgical field, a virtual reality display, an augmented reality display, or a heads-up display.

In Example 50, the subject matter of Examples 43-49 includes, wherein the display device is located on a surgical saw controlling the sawblade.

In Example 51, the subject matter of Examples 43-50 includes, determining whether the detected torque transgressed a threshold, and controlling operation of the surgical saw in response to determining that the detected torque transgressed the threshold.

In Example 52, the subject matter of Example 51 includes, wherein controlling operation of the surgical saw includes causing the sawblade to cease operation in response to determining that the detected torque transgressed the threshold.

In Example 53, the subject matter of Examples 51-52 includes, stopping the sawblade in response to determining that the torque transgressed the threshold.

In Example 54, the subject matter of Examples 51-53 includes, retracting the sawblade in response to determining that the torque transgressed the threshold.

In Example 55, the subject matter of Examples 51-54 includes, blocking the sawblade with a guard component in response to determining that the torque transgressed the threshold.

Example 56 is at least one machine-readable medium including instructions that, when executed by processing circuitry, cause the processing circuitry to perform operations to implement of any of Examples 1-55.

Example 57 is an apparatus comprising means to implement of any of Examples 1-55.

Example 58 is a system to implement of any of Examples 1-55.

Example 59 is a method to implement of any of Examples 1-55.

Method examples described herein may be machine or computer-implemented at least in part. Some examples may include a computer-readable medium or machine-readable medium encoded with instructions operable to configure an electronic device to perform methods as described above. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMS j, read only memories (ROMs), and the like.

What is claimed is:

1. A surgical cutting system comprising:
a surgical saw including a sawblade to perform a surgical cut;
a cut guide used to guide the sawblade while performing the surgical cut;
a sensor to detect torque on the sawblade induced by interaction with the cut guide during the surgical cut; and
a device including a processor and display screen configured to:
receive signals from the sensor indicative of the detected torque; and
generate a visual indication representative of the detected torque for display on the device.

2. The surgical cutting system of claim 1, wherein the device is further configured to determine, based on the received signals, whether the detected torque transgressed a threshold.

3. The surgical cutting system of claim 2, wherein the device is further configured to, in response to determining that the detected torque transgressed the threshold, present a visual indication that the detected torque transgressed the threshold.

4. The surgical cutting system of claim 1, wherein the display screen of the device is located on the surgical saw.

5. The surgical cutting system of claim 1, wherein the sensor is one of an accelerometer, a force sensor, a pressure sensor, or a strain gauge.

6. The surgical cutting system of claim 1, wherein the visual indication representative of the detected torque includes a visual depiction of an orientation of the sawblade relative to the cut guide.

7. A surgical saw comprising:
a sawblade to perform a surgical cut using a cut guide;
a sensor positioned on the sawblade, the sensor to detect torque on the sawblade by the cut guide during the surgical cut;
a processor to control operation of the surgical saw in response to signals from the sensor indicative of torque on the sawblade.

8. The surgical saw of claim 7, wherein the processor is further to determine whether the detected torque transgressed a threshold, and wherein to control operation of the surgical saw, the processor is to control operation of the surgical saw in response to determining that the detected torque transgressed the threshold.

9. The surgical saw of claim 8, wherein to control operation of the surgical saw includes to cause the sawblade to cease operation in response to determining that the detected torque transgressed the threshold.

10. The surgical saw of claim 9, wherein to cause the sawblade to cease operation, the processor is to cause a guard to block the sawblade, cause a motor to stop the sawblade, or to cause the sawblade to be retracted into a shaft of the surgical saw.

11. The surgical saw of claim 8, wherein the processor is further to generate a visual indication representative of the detected torque for display on a display screen.

12. The surgical saw of claim 11, wherein the visual indication includes a representation of the detected torque within a two dimensional torque map.

13. The surgical saw of claim 7, wherein the sensor is one of an accelerometer, a force sensor, a pressure sensor, or a strain gauge.

14. The surgical saw of claim 7, wherein the surgical cut is a training cut performed using the sawblade by a student.

15. The surgical saw of claim 7, wherein the surgical saw further comprises a feedback component to provide feedback indicative of the detected torque.

16. The surgical saw of claim 15, wherein the feedback includes at least one of a visual indication, an auditory indication, or haptic feedback.

17. A method comprising:
detecting, using a sensor, a torque on a robotic arm, the torque caused by a sawblade received within a cut guide attached to the robotic arm;
generating, in response to receiving a signal from the sensor indicative of the torque on the robotic arm, a visual representation of at least a portion of the torque; and
displaying, using a display device, the visual representation of the torque.

18. The method of claim 17, wherein displaying the visual representation of the torque includes displaying a representation of the detected torque within a two-dimensional torque map including displaying torque components representing a medio-lateral detected torque and an anterior-posterior detected torque.

19. At least one non-transitory machine-readable medium including instructions for operation of a surgical saw, which executed by a processor, cause the processor to perform operations to:
detect, using a sensor, a torque on a robotic arm, the torque caused by a sawblade received within a cut guide attached to the robotic arm;
generate, in response to receiving a signal from the sensor indicative of the torque on the robotic arm, a visual representation of at least a portion of the torque; and
display, using a display device, the visual representation of the torque.

20. The at least one machine-readable medium of claim 19, wherein displaying the visual representation of the torque includes displaying an anterior-posterior torque bar corresponding to an anterior-posterior detected torque or a medio-lateral torque bar corresponding to a medio-lateral detected torque.

21. The at least one machine-readable medium of claim 19, wherein displaying the visual representation of the torque includes displaying a representation of the detected torque within a two-dimensional torque map.

22. The at least one machine-readable medium of claim 21, wherein displaying the two-dimensional torque map includes displaying torque components representing a media-lateral detected torque and an anterior-posterior detected torque.

23. The at least one machine-readable medium of claim 21, wherein displaying the two-dimensional torque map includes displaying graduated coloration based on torque level.

24. The at least one machine-readable medium of claim 23, wherein the graduated coloration varies from green to red.

25. The at least one machine-readable medium of claim 19, wherein displaying the visual representation of the torque includes displaying the visual representation on at least one of a display in a surgical field, a virtual reality display, an augmented reality display, or a heads-up display.
